(19) United States
(12) Patent Application Publication
(10) Pub. No.: US 2022/0406406 A1
(43) Pub. Date: Dec. 22, 2022

(54) METHOD AND SYSTEM USING INTEGRATIVE MULTI-OMIC DATA ANALYSIS FOR EVALUATING THE FUNCTIONAL IMPACTS OF GENOMIC VARIANTS

(71) Applicant: KONINKLIJKE PHILIPS N.V., EINDHOVEN (NL)

(72) Inventors: Yee Him CHEUNG, Boston, MA (US); Jie Wu, Cambridge, MA (US); Nevenka Dimitrova, Pelham Manor, NY (US)

(21) Appl. No.: 17/780,037

(22) PCT Filed: Nov. 26, 2020
(86) PCT No.: PCT/EP2020/083444

§ 371 Date: May 26, 2022

Related U.S. Application Data
(60) Provisional application No. 62/940,444, filed on Nov. 26, 2019.

(51) Int. Cl. G16B 20/00; G16B 40/20

(52) U.S. Cl. CPC G16B 20/00; G16B 40/20

(57) ABSTRACT

A method (100) for characterizing a functional impact of a plurality of variants, comprising: obtaining (110) information comprising at least a plurality of variants, gene expression information, copy number variation, and epigenetic effects; determining (120) a splice status for the variant; determining (130) a variant-based expression regulation status, comprising whether the variant has an effect on gene expression; determining (140) a gene-based expression regulation status, comprising an indication of whether the variant has a functional impact on a target gene; determining (150) a gene-based copy number variant (CNV) and epigenetic impact status, comprising whether one or both has an impact on expression of a gene; adjusting (160), based on the CNV and epigenetic impact status, the variant-based and/or the gene-based expression regulation status; and reporting (170) at least the adjusted variant-based and/or the adjusted gene-based expression regulation status for each of a plurality of variants and/or genes from the genomic sample.

### FIELD OF THE DISCLOSURE

The present disclosure is directed generally to methods and systems for improved characterization of the functional impact of genomic variants.

### BACKGROUND

As technology for utilizing different types of molecular information becomes more accessible at a lower cost, it is becoming more common to generate multiple types of -omic data (e.g., genomic, transcriptomic, proteomic, and epigenomic) for the same sample. This allows scientists to better understand the workings of the underlying complex biological system. The launch of commercial assays such as the NanoString® Vantage 3D and the Illumina® TruSight Tumor 170, based respectively on nCounter® and next-generation sequencing (NGS) technologies, which support the simultaneous extraction of DNA and RNA, pushes further the demand for multi-omic data analysis. While the different types of -omic data can be analyzed in separate silos by different bioinformatics pipelines, this mainstream approach fails to take advantage of the underlying inter-relationships across data modalities to build evidence for the functional impact of genomic variants and generate insights on the workings at the molecular level. It also fails to generate new insights into the functional or even pathological impacts of individual aberrations.

### SUMMARY OF THE DISCLOSURE

There is a continued need for methods and systems that evaluate and characterize functional evidence of genomic variants at different levels to provide multi-level evidence for that functional impact. The present disclosure is directed to inventive methods and systems for characterizing the functional impact of a genomic variant. Various embodiments and implementations herein are directed to a system and method that creates a plurality of statuses including a mutation status, a splice variant status, a variant-based expression regulation status, a gene-based expression regulation status, and a gene-based CNV and epigenetic impact status, based on data about variants, gene expression, and other -omic data received by the system. The system utilizes the gene-based CNV and epigenetic impact status to adjust the variant-based expression regulation status and gene-based expression regulation status for the received variants, to produce a final list of variants and associated information through filtering and ranking based on one or more of the generated statuses and scores. A report is generated that includes the finalized list of variants/genes and associated information, including the functional impact(s) of each variant/gene in the finalized list.

Generally, in one aspect, is a method for characterizing a functional impact of a plurality of variants identified from a genomic sample, using a variant analysis system. The method comprises: (i) obtaining genomic sample information, the genomic sample information comprising at least a plurality of variants identified in the genomic sample, gene expression information obtained from the genomic sample, copy number variation for one or more genes in the genomic sample, and epigenetic effects on one or more genes in the genomic sample; (ii) determining a splice status for the variant, the splice status comprising an indication of whether a variant has an effect on splicing of a gene; (iii) determining a variant-based expression regulation status, the variant-based expression regulation status comprising an indication of whether the variant has an effect on expression of a gene; (iv) determining a gene-based expression regulation status, the gene-based expression regulation status comprising an indication of whether the variant has a functional impact on a target gene in the target gene's associated pathway; (v) determining a gene-based copy number variant (CNV) and epigenetic impact status, the gene-based CNV and epigenetic impact status comprising an indication of whether the CNV and/or epigenetic impact has an impact on expression of a gene; (vi) adjusting, based on the gene-based CNV and epigenetic impact status, the variant-based expression regulation status and/or the gene-based expression regulation status; (vii) reporting at least the adjusted variant-based expression regulation status and/or the adjusted gene-based expression regulation status for each of a plurality of variants and/or genes identified in the genomic sample.

According to an embodiment, the method comprises the step of filtering and/or ranking a plurality of variants and/or genes based at least in part on at least the adjusted variant-based expression regulation status and/or the adjusted gene-based expression regulation status information.

According to an embodiment, the splice status further comprises an indication of a strength of splicing evidence for the effect on splicing of the gene.

According to an embodiment, the variant-based expression regulation status further comprises an indication of whether the affected gene is local or distant.

According to an embodiment, the gene-based expression regulation status further comprises an indication of whether the target gene is upregulated or downregulated.

According to an embodiment, the gene-based copy number variant (CNV) and epigenetic impact status further comprises an indication of whether the copy number variant (CNV) and/or epigenetic impact results in potential upregulation or downregulation of a gene.

According to an embodiment, the functional impact information comprises, for one or more of the plurality of remaining variants, an indication of an effect of the variant on the expression of one or more genes.

According to an aspect is a system for characterizing a functional impact of a plurality of variants identified from a genomic sample. The system includes: genomic sample information, the genomic sample information comprising at least a plurality of variants identified in the genomic sample, gene expression information obtained from the genomic sample, copy number variation for one or more genes in the genomic sample, and epigenetic effects on one or more genes in the genomic sample; and a processor configured to: (i) determine a splice status for the variant, the splice status comprising an indication of whether a variant has an effect on splicing of a gene; (ii) determine a variant-based expression regulation status, the variant-based expression regulation status comprising an indication of whether the variant has an effect on expression of a gene; (iii) determine a gene-based expression regulation status, the gene-based expression regulation status comprising an indication of whether the variant has a functional impact on a target gene in a pathway; (iv) determine a gene-based copy number variant (CNV) and epigenetic impact status, the gene-based CNV and epigenetic impact status comprising an indication of whether the CNV and/or epigenetic impact has an impact on expression of a gene; and (v) adjust, based on the gene-based CNV and epigenetic impact status, the variant-based expression regulation status and/or the gene-based expression regulation status.

According to an embodiment, the system further includes a user interface configured to report at least the adjusted variant-based expression regulation status and/or the adjusted gene-based expression regulation status for each of a plurality of variants and/or genes identified in the genomic sample.

According to an embodiment, the system further includes a database that operatively associates the adjusted variant-based expression regulation status with a response to therapy, to a diagnosis, and/or to a prognosis of a patient case.

According to an embodiment, the system further includes a matching algorithm that compares, and/or identifies one or more associations between, the patient genomic profile and the stored associations of the adjusted variant-based expression regulation status with response to therapy, diagnosis, or prognosis of a patient case. According to an embodiment, the system further includes a user interface that reports within a patient context one or more matched associations relevant to the patient at the point of care, wherein the healthcare professional is able to automatically generate a clinical report using these associations.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for characterizing the functional impact of variants in a genomic sample, in accordance with an embodiment.

FIG. 2 is a flowchart of a method for determining a splice status, in accordance with an embodiment.

FIG. 3 is a flowchart of a method for determining a variant-based expression regulation status and/or score, in accordance with an embodiment.

FIG. 4 is a flowchart of a method for determining a gene-based expression regulation status and/or score, in accordance with an embodiment.

FIG. 5 is a flowchart of a method for determining a gene-based CNV and epigenetic impact status and/or score, in accordance with an embodiment.

FIG. 6 is a flowchart of a method for adjusting the variant-based expression regulation status and/or score and the gene-based expression regulation status and/or score, in accordance with an embodiment.

FIG. 7 is a flowchart of a method for characterizing the functional impact of variants in a genomic sample, in accordance with an embodiment.

FIG. 8 is a schematic representation of a variant analysis system, in accordance with an embodiment.

### DETAILED DESCRIPTION OF EMBODIMENTS

The present disclosure describes various embodiments of a system and method to more accurately determine the functional impact of variants and genes, identified in a sample, on gene expression. More generally, Applicant has recognized and appreciated that it would be beneficial to provide a method that characterizes in detail a functional impact of a variant. The system determines: (i) a splice status for the variant; (ii) a variant-based expression regulation status comprising on indication of whether the variant has an effect on expression of a gene; and (iii) a gene-based expression regulation status comprising an indication of whether the variant has a functional impact on a target gene in a pathway. The system also determines a gene-based copy number variant (CNV) and epigenetic impact status, comprising an indication of whether the CNV and/or epigenetic impact has an impact on expression of a gene. The system uses the gene-based CNV and epigenetic impact status to adjust the variant-based expression regulation status and/or the gene-based expression regulation status. The adjusted variant-based expression regulation status and gene-based expression regulation status comprises the information about the functional impact of the variants and genes. This functional impact information, and other information, can then be reported out for one or more variants and/or genes.

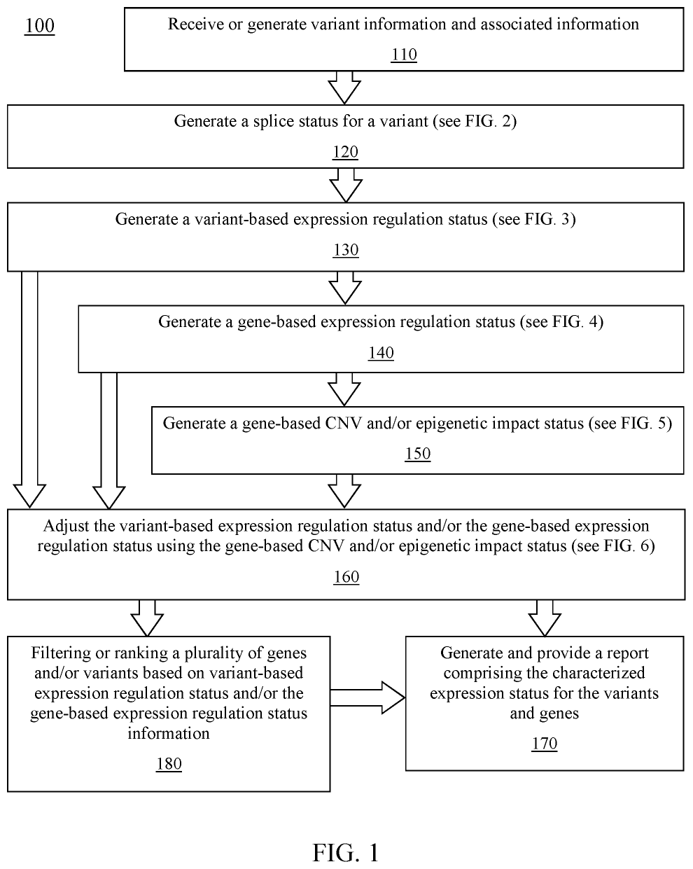

Referring to FIG. 1, in one embodiment, is a flowchart of a method 100 for characterizing variant expression status of variants in a genomic sample using a variant analysis system. The variant analysis system may be any of the systems described or otherwise envisioned herein, and may comprise any of the components described or otherwise envisioned herein.

At step 110 of the method, the variant analysis system generates and/or receives DNA and RNA sequencing data for a genetic sample. The genetic sample can be any genetic sample from any organism, including humans, pathogenic and non-pathogenic organisms, and many. It is recognized that there is no limitation to the source of the genetic sample.

According to an embodiment, the variant analysis system comprises a DNA and/or RNA sequencing platform configured to obtain sequencing data from the genetic sample. The sequencing platform can be any sequencing platform, including but not limited to any system described or otherwise envisioned herein. A sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner. According to an embodiment, the variant analysis system receives the DNA and/or RNA sequencing data for the genetic sample. For example, the variant analysis system may be in communication or otherwise receive DNA and/or RNA sequencing data from a database comprising one or more genetic samples.

The generated and/or received DNA and/or RNA sequencing data may be stored in a local or remote database for use by the variant analysis system. For example, the variant analysis system may comprise a database to store the DNA and/or RNA sequencing data for the genetic sample, and/or may be in communication with a database storing the sequencing data. These databases may be located with or within the variant analysis system or may be located remote from the variant analysis system, such as in cloud storage and/or other remote storage.

The generated and/or received DNA and/or RNA sequencing data may comprise a complete or mostly complete genome, or may be a partial genome, or may be a small portion of a genome. For example, the generated and/or received sequencing data may be assemblies, whole genome constructs, incomplete genomes, partial genomes, exomes, and/or any other sequencing data.

The generated and/or received DNA and/or RNA sequencing data each comprise a plurality of different variant types, including but not limited to single nucleotide variants, insertions, deletions, copy number variants, and gene fusions. Many other variant types are possible. Gene fusions may be detected using a variety of systems, including but not limited to dRanger with Breakpointer, FusionMap, and/or other tools. Other structural variants such inversions, translocations, and others may be detected using a variety of systems, including but not limited to SVDetect, BreakDancer, and/or other tools.

The generated and/or received RNA sequencing data also comprises expression data for each variant, including but not limited to gene expression data, transcript expression data, exon expression data, splicing data, and/or allele-specific expression data. The expression data is obtained, analyzed, reported, and/or stored using any method utilized to do so from RNA sequencing data. The expression data can comprise information about allele-specific expression (ASE); allele-specific splicing (ASS); exon, transcript and gene (including long non-coding RNA, i.e. lncRNA) expressions; differential exon, transcript and gene (including lncRNA) expressions, either based on comparison with a matched normal sample and/or average expressions and their standard deviations in unrelated normal tissues; and/or gene pathway activity prediction by running methods such as Philips OncoSignal and other methods on gene expression and other required data.

If the source is a germline, obtained data may include the genotype (such as homozygous major, heterozygous, homozygous minor), copy number (which could be compared with healthy population of the same background), and/or other information. If the source is somatic, obtained data may include variant allele frequency (VAF), differential copy number variation (compared with matched or unrelated normal tissues), and/or other information.

At step 120 of the method, the system generates a splice status for a variant, the splice status comprising a type of splicing effect of the variant. This is effectively a variant-based splicing regulatory status and score. For example, the splice status may comprise a predefined or user-defined variable that indicates that the variant has no splicing effect, or a splicing effect indicating that the variant only affects splicing of a gene local to the variant where ‘local’ may be for example a predefined or user-defined range using centiMorgans or megabase pairs, among other ranges or location-based definitions. For example, local may be defined as the gene or genes immediately on either side of the variant, or the gene within which the variant is located if it is located within a gene. The splice status may comprise a splicing effect indicating that the variant only affect splicing of a distant gene where ‘distant’ may be may be for example a predefined or user-defined range using centiMorgans or megabase pairs, among other ranges or location-based definitions. For example, distant may be defined as gene or genes not immediately on either side of the variant, or genes other than the one within which the variant is located if it is located within a gene. The splice status may comprise a cis and trans splicing effect, and may indicate that the variant affects splicing of a local and a distant gene.

According to an embodiment, the splice status also comprises an indication of the strength of the splicing evidence. For example, the indication of the strength of the splicing evidence can comprise the type of supporting evidence for the splicing effect, which can be “allele_specific_splicing,” “differential exon expression,” “differential transcript expression,” or other applicable types. The indication may be a score indicating the strength of the splicing evidence such as the log 2 fold change between the allele-carrying and wild-type reads, fold change in exon/transcript expressions, or another indication.

According to an embodiment, the splice status comprises a chart, table, or other summary of information comprising the variant identification, the type of splicing effect for the variant, the type of supporting evidence for the splicing effect resulting from the variant, and/or a score for the strength of the supporting evidence for the splicing effect.

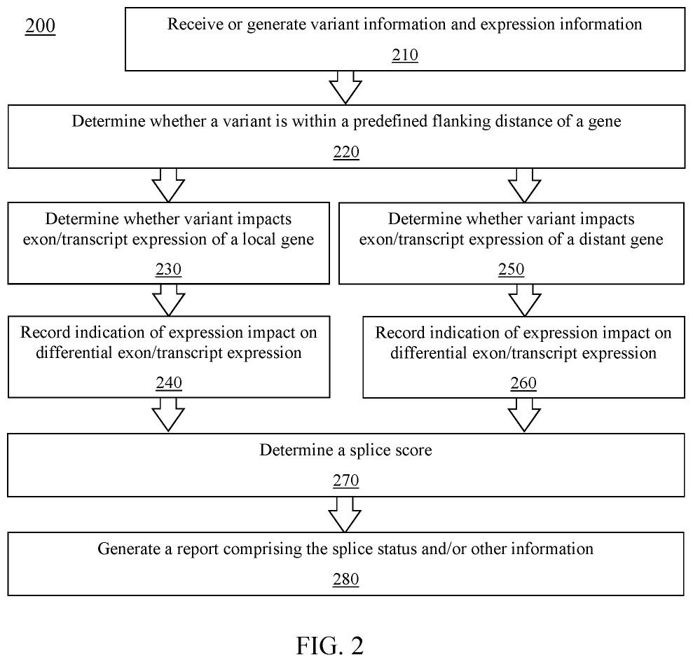

Referring to FIG. 2 is a flowchart of a method for generating a splice status for a variant. At step 210 of the method, the system generates or receives a list of variants identified in a genomic sample, along with associated expression information comprising one or more of differential exon/transcript expression between allele carriers and non-carriers (or change in splicing ratios, or other similar measures), and allele-specific splicing data.

At step 220 of the method, the system determines for one or more variants in the list of variants whether the variant is located within a defined flanking distance of the 5′ and 3′ ends of the i^{th} exon (exon_i) of a gene (gene_x), where the defined flanking distance is predefined or user-defined. For example, a user may define a flanking distance based on preference and/or experimentation, the flanking distance may be determined by a programmer, or the flanking distance may be defined using any other process or setting.

At step 230 of the method, the system analyzes the received differential exon expression, differential transcript expression, and/or allele-specific splicing data to determine whether the variant impacts the expression of a local gene. For example, if the variant demonstrates allele-specific splicing of a local gene, then the system records that indication at step 240. As just one example, the system can register the indication in a table or other data entry form that there is allele-specific splicing of a local gene (such as “Cis”, “gene_x:exon_i”, “allele_specific_splicing”, and value, although many other variations are possible). If the variant results in differential exon expression, then the system records that indication at step 240. As just one example, the system can register the indication in a table or other data entry form that there is differential exon expression of a local gene (such as “Cis”, “gene_x:exon_i”, “differential_exon_expression”, and value, although many other variations are possible). If the variant results in differential transcript expression, then the system records that indication at step 240. As just one example, the system can register the indication in a table or other data entry form that there is differential transcript expression or a local gene (such as “Cis”, “gene_x:exon_i”, “differential_transcript_expression”, and value, although many other variations are possible).

At step 250 of the method, the system determines whether the variant impacts exon/transcript expression of a distant gene. To do this, the system searches a database such as a sQTL (splicing quantitative trait loci) database to determine whether the variant is associated with an impact on a distant gene. The database may comprise cis information (cis-acting regulation of alternative splicing in a nearby gene) and/or trans information (trans-acting regulation of alternative splicing in a distant gene). If the variant is found to be associated with an impact on a distant gene, the association is recorded in a in a table or other data entry form at step 260 (such as “Trans”, “target_gene_x”, “differential_transcript_expression”, and value, although many other variations are possible).

At step 270 of the method, a score is determined. If there is no indication that the variant has an effect on splicing, then a score such as “none” or “0” is recorded, or alternatively nothing is recorded. If there is an indication that the variant does have an effect on splicing, then a score is calculated for the strength of the evidence supporting the splicing effect. For example, the score may comprise the log 2 fold change between the allele-carrying and wild-type reads, the fold change in exon/transcript expressions, or any other indication of the effect of splicing caused by the variant.

At step 280 of the method, a splice status and/or splice score are reported, such as via a data table or other data format, or via a printed or displayed report. According to an embodiment, the report comprises one or more of:
A splice status (splice_status) which is a categorical variable that indicates the type of splicing effect of a variant as “Cis” (only affect splicing of a local gene), “Trans” (only affect splicing of a distant gene), “Cis and Trans” (both local and distant splicing influence), and/or “None” (no splicing effect);
A splice score (splice_score) which is a score measuring the strength of splicing evidence. The splice_score can be a function of splice_results, such as choosing the maximum normalized evidence_value; and
A splice data structure (splice_results) comprising a table or other data structure or format summarizing the splicing effects of a variant with one or more of the following fields, among other possible fields:
   type—the type of splicing effect, which can be “Cis” (on a local gene) or “Trans-sQTL” (on a distant gene, based on reported splice sites in sQTL databases);
   target—the target of the splicing action, which can be a specific gene exon for cis-splicing or just the target gene for trans-splicing;
   evidence_type—the type of supporting evidence for the splicing effect, which can be “allele_specific_splicing”, “differential exon expression”, “differential transcript expression”, or other applicable types; and/or
   evidence_value—a value that measures the strength of the supporting evidence. Depending on the evidence type, it can be the log 2 fold change between the allele-carrying and wild-type reads, fold change in exon/transcript expressions, etc.

At step 130 of the method depicted in FIG. 1, the system generates a variant-based expression regulation status. This is effectively an analysis of the variant on the regulation of expression of one or more local and/or distant genes. For example, the variant-based expression status may comprise a predefined or user-defined variable that indicates that the variant has no effect on the regulation of expression, upregulation of a local and/or distant gene, and/or downregulation of a local and/or distant, among other indications.

According to an embodiment, the goal is to evaluate the functional evidence for expression regulation of each genomic variant that is either in the promoter/enhancer of a gene (cis-acting—promoter/enhancer), or reported in external eQTL databases to regulate the expression of a local/distant gene (cis/trans-acting-eQTL). Another example of a database is the EPDnew (Eukaryotic Promoter Database), although other sources are possible.

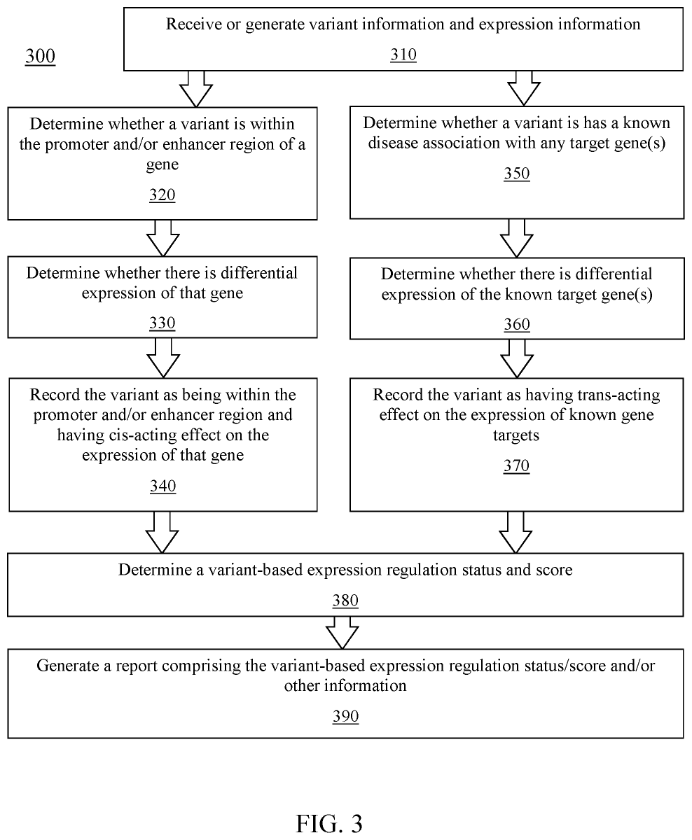

Referring to FIG. 3 is a flowchart of a method for generating a variant-based expression regulation status. At step 310 of the method, the system generates or receives a list of variants identified in a genomic sample. The system also generates or receives differential gene (optionally including lncRNA) expression information.

At step 320 of the method, the system first determines for one or more variants in the list of variants whether the variant is located within the promoter region of a gene (gene_x), where the location of a promoter region may be predefined or user-defined. For example, the user-defined region may comprise a user-defined upstream distance from the transcription start site. Alternatively, the predefined region may be based on known/predicted promoters in a database. Accordingly, the system may comprise a promoter database or be in contact with a promoter database.

If the variant is not located within the promoter region, the system determines whether the variant is within the enhancer region of the gene (gene_x), where the location of an enhancer region may be predefined in an enhancer database such as the FANTOM5 (Functional ANnoTation Of the Mammalian genome), although other sources are possible.

At step 330 of the method, the system determines whether there is differential expression of the gene (gene_x) between the allele carriers and non-carriers, using the received or generated differential gene (optionally including lncRNA) expression information. If there is differential expression of gene (gene_x) and the variant is located in a promoter and/or enhancer region, then the system records that indication at step 340. As just one example, the system can register the indication in a table or other data entry form var_reg_results (such as “Cis-Promoter”, “gene_x”, “differential_gene_expression”, and value, although many other variations are possible).

At step 350 of the method, the system further determines whether the variant is known to be associated with the differential expression of one or more target genes (gene_x) and the direction (reg_dir_x) of that differential expression (up or down regulation). For example, the system may utilize an expression quantitative trait loci (eQTL) database such as the GTEx (Genotype-Tissue Expression) eQTL database, and other sources are possible.

At step 360 of the method, the system determines whether there is observed differential expression of the gene (gene_x) between the allele carriers and non-carriers, using the received or generated gene (optionally including lncRNA) expression information, in the same direction (reg_dir_x) as the direction from the expression database. If there is differential expression of the target gene (gene_x) in the same direction (reg_dir_x) as the direction from the expression database, then the system records that indication at step 370. As just one example, the system can register the indication in a table or other data entry form var_reg_results (such as “[Cis/Trans]-eQTL”, “gene_x:reg_dir_x”, “differential_gene expression”, and value, although many other variations are possible).

At step 380 of the method, variant-based expression regulation status and score are determined. If there is no indication that the variant has any effect on the regulation of expression, then as the status can be recorded as “none” and the score as “0.” If there is an indication that the variant does have an effect on the regulation of expression, then a score is calculated for the strength of the evidence supporting the effect of the variant on the regulation of expression. For example, the score may be based on the target gene with the largest magnitude of expression change resulting from regulation, regardless of the sign/direction of the expression change.

At step 390 of the method, a variant-based expression regulation status and/or variant-based expression regulation score is reported, such as via a data table or other data format, or via a printed or displayed report. According to an embodiment, the report comprises one or more of:
A variant-based expression regulation status (var_reg_status) which is a categorical variable that indicates the type of expression regulatory effect of a variant as “Cis-Promoter” (cis-acting and in the promoter region of a local gene), “Cis-Enhancer” (cis-acting and in the enhancer of one or more genes), “Trans-eQTL” (trans-acting as defined in eQTL databases), “Cis and Trans” (both cis- and trans-acting gene expression regulations), and/or “None” (no expression regulatory effect);
A variant-based expression regulation status score (var_reg_score) which is a score that measures the strength of gene expression regulatory evidence. The score can be a function of var_reg_results, such as choosing the evidence_value with the largest magnitude (regardless of the sign/direction); and
A variant-based expression regulation status data structure (var_reg_results) comprising a table or other data structure or format summarizing the gene expression regulatory effects of a variant with one or more of the following fields, among other possible fields:
   type—the type of regulatory effect, which can be “Cis-Promoter,” “Cis-Enhancer,” “Trans-eQTL,” “Cis and Trans,” or “None”;
   target—the target of the regulatory action, which can be the symbol of the affected gene, optionally concatenated by “:” followed by the regulatory direction (up/down) if available;
   evidence_type—the type of supporting evidence for the regulatory effect, which can be “differential_gene_expression” or other applicable types; and/or
   evidence_value—a value that measures the strength of the supporting evidence. For evidence based on differential expression, it can be the log 2 fold change of case vs. control expression levels, among other values.
      Many other fields and data are possible.

At step 140 of the method depicted in FIG. 1, the system generates a gene-based expression regulation status. This is effectively an analysis of gene-gene interactions to determine whether a gene has a functional impact on a target gene in a pathway. For example, the gene-based expression regulation status may comprise a variable that indicates the strongest type of expression regulatory effect of a gene on its direct gene targets defined in the pathway databases. According to an embodiment, the goal is to evaluate the functional evidence for each gene-gene interaction as identified by differential expression between cases and controls, or disease versus normal tissue samples, either collectively or per individual matched sample pairs, and as defined in external pathway databases.

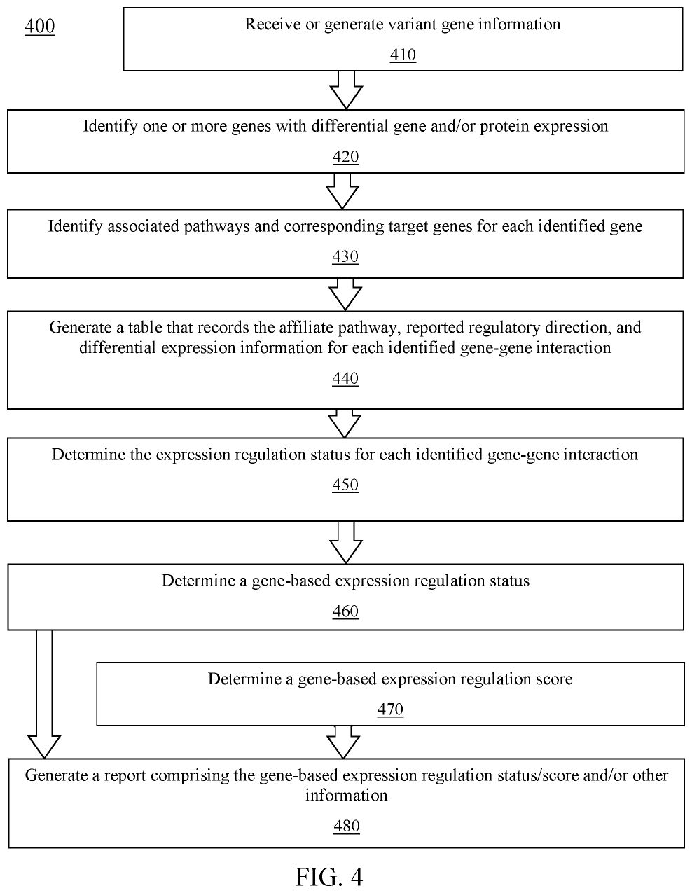

Referring to FIG. 4 is a flowchart of a method for generating a gene-based expression regulation status. At step 410 of the method, the system generates or receives differential gene (optionally including lncRNA) expression information, and/or differential protein expression.

At step 420 of the method, the system identifies one or more genes with differential gene expression based on the generated or received RNA-seq and/or differential protein expression based on proteomic data. Depending on the study hypothesis, different selection strategies can be applied. For example, one may select for genes showing significant differential expression between the groups of disease and normal samples collectively, or genes that are significantly differentially expressed (in both up or down directions) in more than a certain number/percentage of individual matched disease-normal sample pairs. In the subsequent discussions, all examples are given based on the first scenario where collective differential expression is concerned, although this does not limit the different scenarios or selection strategies that may be utilized per this method.

At step 430 of the method, the system identifies associated pathways and corresponding gene targets from one or more pathway databases for each of the identified genes. The pathway database may be any database with gene pathway information, including but not limited to KEGG, Reactome, Pathway Commons, and others.

At step 440, a gene-gene regulation table gene_reg_results is generated to capture information such as the affiliated pathway, reported regulatory direction, and observed differential gene expression in the data. For example, gene_1 and gene_2 can be labels of the ‘from’ (gene_1) and ‘to’ (gene_2) genes of an edge in the pathway (path) found in the pathway database (path_db). And de_1 (or de_2), de_status_1 (or de_status_2) can be respectively the differential expression value (e.g. in log 2 fold change) and status (up, down, or none) for gene_1 (or gene_2)

At step 450, the system determines the expression regulation status of each gene-gene interaction. If the downstream gene is differentially expressed and the upstream gene (gene_1) is not differentially expressed, then the status (status) is recorded as non-differentially expressed. For example, a label such as “Non-DE” indicates there is non-differential expression originated expression regulation. Indeed, genes not differentially expressed can still influence its downstream target if its protein function is altered.

Similarly, if it is not defined in the pathway database or it is an unknown status, then the status (status) is recorded as being an unknown direction. For example, a label such as “Unknown Direction” indicates there is unknown regulatory direction.

If the directions of differential expression of both the upstream gene (gene_1) and the downstream gene (gene_2) agree with the predefined regulatory direction in the database, then the status (status) is recorded as such. For example, a label such as “Agreed Direction” for the status indicates the differential expression of both genes agree with the known information.

If the directions of differential expression of the upstream gene (gene_1) and the downstream gene (gene_2) fail to agree with the predefined regulatory direction in the database, then the status (status) is recorded as such. For example, a label such as “Opposite Direction” for the status indicates the differential expression of one or both genes does not agree with the known information.

According to an embodiment, the gene-gene regulation status is recorded in a table or other data format or structure. For example, the status may comprise the format (“Path_db:path,” gene_1, gene_2, status, de_1, de_status_1, de_2, de_status_2), along with many other possible formats.

At step 460, an overall expression regulation status is determined for each identified gene based on the gene-gene regulation table gene_reg_results generated in steps 440 and 450. According to an embodiment, for gene g, the system finds all entries in gene_reg_results matched by gene_1. If there is no matching entry, then the gene_reg_status=“No Evidence.” Otherwise, if there are any matching entries with status “Agreed Direction”, then the gene_reg_status=“Agreed Direction.” Otherwise, if there are any matching entries with status “Unknown Direction”, then the gene_reg_status=“Unknown Direction.” If there are any matching entries with status “Non-DE”, then the gene_reg_status=“Non-DE.” Otherwise, gene_reg_status=“Opposite Direction.”

At step 470 of the method, the system generates one or more gene-based expression regulation scores. For example, the system may generate one or a vector of scores (gene_reg_score_close) that quantify the evidence for expression regulatory effect of a gene on its immediate or close targets. According to an embodiment the system may use the numbers of direct targets of a gene in each type of regulatory status, namely “Agreed Direction”, “Unknown Direction”, “Non-DE” and “Opposite Direction” as recorded in the gene-gene regulation table. As another example, the system may generate one or a vector of scores (gene_reg_score_ext) that quantify the evidence for expression regulatory effect of a gene on its extended downstream targets, up to a user-defined distance d (in number of genes). According to an embodiment the system may use the numbers of extended targets in each type of regulatory status, namely “Agreed Direction”, “Unknown Direction”, “Non-DE” and “Opposite Direction”.

According to an embodiment, these numbers of direct targets and/or extended targets may be determined with the following, although other approaches are possible:
Let g_fr and p be respectively vectors of genes and the corresponding pathways;
g_fr=g; p=null; tab=null;
for i=1 to d:
   If p==null, then t=all entries in gene_reg_results where gene_1 matches with any of the elements in g_fr;
   If p< >null, then t=all entries in gene_reg_results where both gene_1 and path match respectively with g_fr and p at same vector position;
   Remove any entries in t that are already in tab;
   g_fr=gene_2 in t; p=path in t;
   Append t to tab; then
Compute n_agr_e, n_unk_e, n_nde_e and n_opp_e by counting the number of entries in tab of status “Agreed Direction”, “Unknown Direction”, “Non-DE” and “Opposite Direction” respectively.

At step 480 of the method, a gene-based expression regulation status and/or gene-based expression regulation score is reported, such as via a data table or other data format, or via a printed or displayed report. According to an embodiment, the report comprises one or more of:
A gene-based expression regulation status (gene_reg_status) which is a categorical variable that indicates the strongest type of expression regulatory effect of a gene on its direct gene targets defined in the pathway databases. Possible categories include, but are not limited to:
   “Agreed Direction”—observed differential expressions of the gene and its downstream target in agreement with the defined regulatory direction;
   “Unknown Direction”—both the gene and its downstream target are differentially expressed, but the regulatory direction is undefined;
   “Non-DE”— the target gene is differentially expressed but not the upstream gene;
   “Opposite Direction”—the observed differential expressions of the up- and downstream genes are opposite to the defined regulatory direction; and/or
   “No Evidence”—no differential expression observed in any of the target genes.
A gene-based expression regulation score for close targets (gene_reg_score_close) which is one or a vector of scores that quantify the evidence for expression regulatory effect of a gene on its immediate or close targets;
A gene-based expression regulation score for extended downstream targets (gene_reg_score_ext) which is one or a vector of scores that quantify the evidence for expression regulatory effect of a gene on its extended downstream targets, up to a user-defined distance d (in number of genes);
A gene-based expression regulation status data structure (gene_reg_results) comprising a table or other data structure or format summarizing the gene-based expression regulation status with one or more of the following fields, among other possible fields:
   path_db:path—the pathway database and the gene pathway in which the gene-gene regulation is defined;
   gene_1—the upstream gene;
   gene_2—the direct downstream target gene;
   status—the type of evidence for the gene-gene regulation;
   de_1—differential expression value, e.g. in log 2 fold change, of gene_1;
   de_status_1—differential expression status, i.e. up/down, of gene_1;
   de_2—differential expression value, e.g. in log 2 fold change, of gene_2; and/or
   de_status_2—differential expression status, i.e. up/down, of gene_2.
      Many other fields and data are possible.

At step 150 of the method depicted in FIG. 1, the system generates a gene-based copy number variant (CNV) and/or epigenetic impact status and/or score. This is an analysis of the CNV and epigenetic influence on each gene. For example, gene-based copy number variant (CNV) and epigenetic impact status may comprise a categorical value that indicates the combined CNV and epigenetic effect on the gene expression. According to an embodiment, the goal is to evaluate the CNV and epigenetic influence on each gene.

Although only three factors (CNV, methylation, and transcription factor binding) are discussed below, the method can comprise additional epigenetic factors or any other factors by weighting in their effects in a similar fashion.

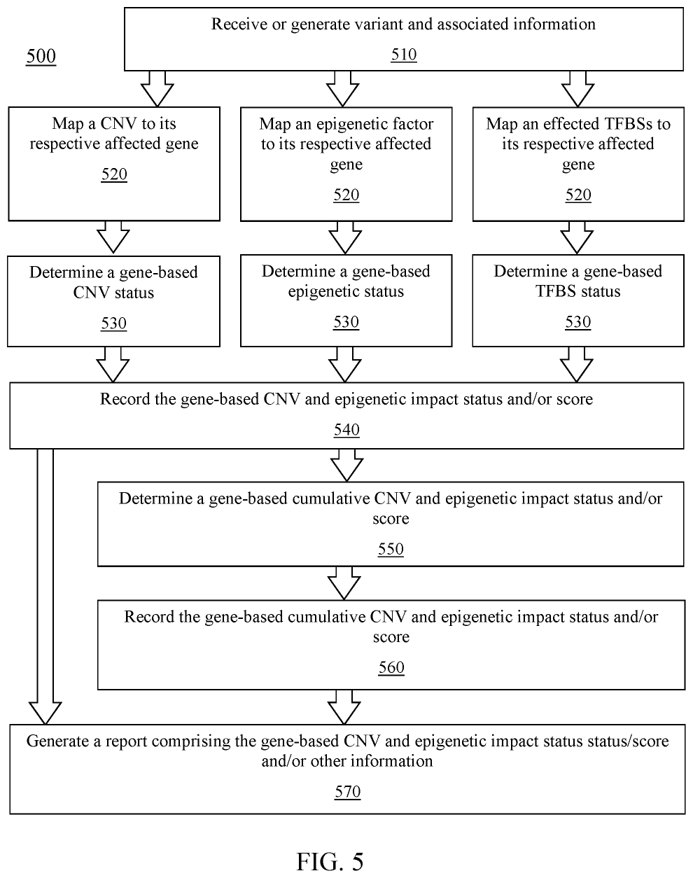

Referring to FIG. 5 is a flowchart of a method for generating a gene-based copy number variant (CNV) and epigenetic impact status and/or score. At step 510 of the method, the system generates or receives a list of variants identified in a genomic sample. The system also generates or receives information about copy number variation, differential methylation at gene promoters, and/or differential binding (e.g. read-enrichment fold changes) at transcription factor binding sites (TFBS). This information about CNVs, epigenetic factors, and differential binding is obtained by any CNV and epigenetic analysis known now or in the future. These analyses are performed from the same genomic source that provided the variants identified in a genomic sample. The system also generates or receives differential gene expression information and/or differential protein expression.

According to an embodiment, a CNV, epigenetic factor, and/or differential binding at a TFBS may be identified by comparing the results of the analysis on the genomic source to a database of known CNVs, epigenetic factors, and/or differential binding at a TFBS (such as the GTRD (Gene Transcription Regulation Database) among other possible databases), and/or to a comparative genome source or sample. For example, the original genomic source may be a tumor sample, while the comparative source or sample may be a non-tumor sample from the same individual.

At step 520 of the method, the system maps one or more CNVs received at step 510 to the corresponding gene affected by that CNV, based on the genomic coordinates of the identified CNV. For example, the corresponding gene may be a gene within which the CNV is located. Alternatively or additionally, the system maps an epigenetic factor received at step 510 to the corresponding gene affected by that epigenetic factor based on the genomic coordinates of the identified epigenetic factor. For example, the corresponding gene may be a gene with a promoter having a differentially methylated site. Alternatively or additionally, the system maps a TFBS with differential binding to the corresponding gene affected by that differential binding, based on the genomic coordinates of the identified TFBS. For example, the corresponding gene may be a gene with a TFBS that overlaps the differential binding site obtained by peak calling in ChIP-Seq data.

At step 530, each gene is analyzed to determine a gene-based CNV, epigenetic, or TFBS impact status and/or score. For example, a gene identified as being affected by a CNV is analyzed to determine the gene-based CNV status and/or score. A gene identified as being affected by an identified epigenetic factor is analyzed to determine the gene-based epigenetic factor status and/or score. A gene identified as being affected by an identified TFBS differential binding is analyzed to determine the gene-based TFBS differential binding status and/or score. According to an embodiment, each gene identified as being affected by a CNV is analyzed to determine whether CNV expression is upregulated, down regulated, or neutral relative to a comparative source or sample, or database. Similarly, each gene identified as being affected by an epigenetic factor is analyzed to determine whether epigenetic modification is upregulated, down regulated, or neutral relative to a comparative source or sample, or database. Similarly, each gene identified as being affected by TFBS differential binding is analyzed to determine whether TFBS differential binding is upregulated, down regulated, or neutral relative to a comparative source or sample, or database. There are many mechanisms, processes, and algorithms that can be utilized to determine the gene-based CNV, epigenetic, or TFBS impact status and/or score.

According to an embodiment, the gene-based CNV, epigenetic, or TFBS impact status and/or score is determined according to the following steps. This method is provided as an example only, and does not limit the scope of this method. According to the method, one or more of the following parameters are defined
Define cnv_hi and cnv_lo as the user-defined upper and lower bounds of the differential copy number value (they can be an absolute or percentile (with reference to the background) value);
   Define meth_hi and meth_lo as the user-defined upper and lower bounds of the differential methylation value (they can be an absolute or percentile (with reference to the background) value);
   Define bind_hi and bind_lo as the user-defined upper and lower bounds of the TFBS differential binding value (they can be an absolute or percentile (with reference to the background) value);
Define k_cnv, k_meth and k_bind as user-defined weightings for the effect on gene expressions due to CNV, methylation, and transcription factor binding respectively; and
Define cnv_epi_hi and cnv_epi_lo as the user-defined upper and lower bounds of the combined CNV and epigenetic effect on gene expression (they can be an absolute or percentile (with reference to the background) value).

With these parameters, the system can determine the status and/or score for a gene affected by CNV, epigenetic factor, and/or TFBS differential binding using the following or similar steps, per this embodiment:
Assign or compute values for cnv_value, meth_value and bind_value, such as by averaging the log 2 fold changes over multiple sites;
For a gene affected by CNV, if the cnv_value>cnv_hi, then the cnv_effect=“Up”; else if cnv_value<cnv_lo, then cnv_effect=“Down”; Else cnv_effect=“Neutral”;
For a gene affected by epigenetic factor, if meth_value<meth_lo, then meth_effect=“Up”; else if meth_value>meth_hi, then meth_effect=“Down”; ese meth_effect=“Neutral” (notably, since DNA methylation represses transcription, a low (high) differential methylation means up-regulation (down-regulation) of gene expression); and
For a gene affected by TFBS differential binding, if bind_value>bind_hi, then bind_effect=“Up”; Else if bind_value<bind_lo, then bind_effect=“Down”; Else bind_effect=“Neutral”.

At step 540 of the method, the system records the status and/or score in a table or other data entry form.

According to an embodiment, the status can result in activation of an oncogene or inactivation of the tumor suppressor gene. In a cancer sample, either one of these results may be important as it may be associated with information on diagnosis, prognosis, or response to therapy. In a germline sample, this information may be associated with a predisposition to cancer.

Oncogenes encode proteins that drive cell proliferation and programmed cell death. Oncogenes are divided into six different classes: transcription factors, proteins remodeling chromatin structure, growth factors, growth factor receptors, signal transducers of signaling pathways, and apoptosis regulators. Oncogenes can be activated by mutations, amplifications, or rearrangements (fusions).

One such example is the epidermal growth factor receptor (EGFR), which is a receptor tyrosine kinase belonging to the HER family of receptor tyrosine kinases. Receptor activation upon ligand binding leads to downstream activation of the PI3K/AKT, RAS/RAF/MEK/ERK and PLCγ/PKC pathways. These pathways have an influential role in cell proliferation, survival, and the metastatic potential of tumor cells. Increased activation by gene amplification, protein overexpression, or mutations of EGFR has been identified as an etiological factor in a number of human epithelial cancers including non-small cell lung cancer, colorectal cancer glioblastoma, and breast cancer. For example, if there is copy number variation that results in ERBB2 amplification, then it may result in activation and this active form is associated with response to trastuzumab or pertuzumab. There are a number of drugs that target these activated forms, and there exists published clinical evidence for conferring response to these drugs when an activating mutation or amplification is detected.

| Drug target | Drug | Biomarker | Indication |
|---|---|---|---|
| EGFR | Afatinib | EGFR L858R, EGFR | NSCLC |
| | | exon 19 deletion | |
| EGFR | Cetuxumab | EGFR positive, | Colorectal cancer |
| | | KRAS wildtype | |
| BRAF | Dabrafenib | BRAF V600E, BRAF | Melanoma |
| | | V600K | |

CNV is known to be associated with many diseases. As just one example, having more copies of oncogenes may increase the risk of disease. However, if the promoter of the oncogene is hyper-methylated, the risk could be offset by the repressive effect on the oncogene. Similarly, having more copies of tumor-suppressor genes may reduce the risk of disease. However, if the promoter of the tumor-suppressor gene is hyper-methylated, the protection effect could be counteracted. Therefore, it is important to consider the combined impact of CNV and epigenetic factors in clinical applications as supported by the methods and systems for multi-omic data analysis described or otherwise envisioned herein.

Yet another application of the methods and systems for multi-omic data analysis described or otherwise envisioned herein is to utilize SNV data to confirm copy-neutral loss of heterozygosity. These segments have a normal copy number of two. However, the two copies are identical to each other resulting in the same clinical impact as copy number loss. Such events could only be detected by analyzing SNV and CNV data together.

At optional step 550 of the method, the system determines a cumulative status and/or score for gene-based CNV, epigenetic factor, and/or TFBS differential binding impact or effect. This can be accomplished by, for example, summing or otherwise combining or processing the statuses or values for CNV impact, epigenetic factor impact, and/or TFBS differential binding impact or effect. The cumulated score may be any combination of two or more of the CNV impact, epigenetic factor impact, and TFBS differential binding.

According to one non-limiting example, the system utilizes the following equations or algorithm to determine the cumulative status and/or score for the CNV and epigenetic factor impact:
cnv_epi_value=k_cnv*cnv_value−k_meth*meth_value+k_bind*bind_value; and
If the calculated cnv_epi_bind_value>cnv_epi_hi, then cnv_epi_effect=“Up”; else if the cnv_epi_value<cnv_epi_lo, then cnv_epi_effect=“Down”; else cnv_epi_effect=“Neutral”.

At step 560 of the method, the system records the determined cumulative status and/or score in a table or other data entry form.

At step 570 of the method, the determined statuses and/or scores are reported, such as being stored in a data table or other data format, or via a printed or displayed report. According to an embodiment, the report comprises one or more of, for each gene in the analysis:
A status of the CNV effect (cnv_effect) which can be a categorical value that indicates the effect of the CNV on the gene expression. Possible categories or values include, but are not limited to: “Up” for upregulation of gene expression, “Down” for downregulation of gene expression, and “Neutral” for no significant change in gene expression;
A score for the CNV effect (cnv_value) which can be a differential copy number value (log 2 fold change, with respect to matched normal tissue and/or healthy population of the same ethnicity/generic baseline of 2);
A status of the epigenetic effect (meth_effect) which can be a categorical value that indicates the effect of the epigenetic factor on the gene expression. Possible categories or values include, but are not limited to: “Up” for upregulation of gene expression, “Down” for downregulation of gene expression, and “Neutral” for no significant change in gene expression;
A score for the epigenetic effect (meth_value) which can be a differential methylation value (log 2 fold change). Although described with regard to methylation, other epigenetic factors are possible;
A status of the TFBS binding (bind_effect) which can be a categorical value that indicates the transcription factor binding effect (such as due to histone modifications) on the gene expression. Possible categories or values include, but are not limited to: “Up” for upregulation of gene expression, “Down” for downregulation of gene expression, and “Neutral” for no significant change in gene expression;
A score for the TFBS binding (bind_value) which can be a differential binding value (log 2 fold change)
A cumulative status indicating an effect on gene expression (such as cnv_epi_effect) which is a categorical value that indicates the combined CNV, epigenetic, and/or TFBS effect on the gene expression. Possible categories or values include, but are not limited to: “Up” for upregulation of gene expression, “Down” for downregulation of gene expression, and “Neutral” for no significant change in gene expression; and/or
A cumulative quantitative score (such as cnv_epi_value) that measures the combined CNV, epigenetic, and/or TFBS effect on the gene expression.
   Many other fields and data are possible.

At step 160 of the method depicted in FIG. 1, the system generates a CNV and epigenetic factor-adjusted expression regulation status and/or score. This is a re-evaluation of the variant-based expression regulation status and score from step 130 of the method and/or a re-evaluation of the gene-based expression regulation status and score from step 140 of the method, by adjusting for the CNV and epigenetic factors from step 150 of the method.

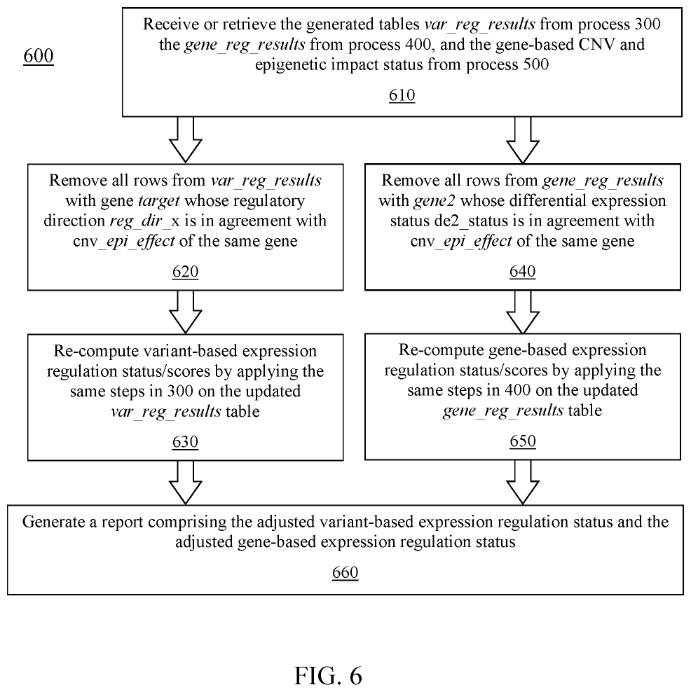

Referring to FIG. 6 is a flowchart of a method for generates a CNV and epigenetic factor-adjusted expression regulation status and/or score. At step 610 of the method, the system receives or otherwise retrieves one or more of: (1) the generated variant-target regulation table var_reg_results from steps 330-360 of the method; (2) the generated gene-gene regulation table gene_reg_results from steps 440-450 of the method; and (3) the gene-based copy number variant (CNV) and epigenetic impact status from step 150 of the method. For example, the gene-based CNV and epigenetic impact status from step 150 of the method could be the cnv_epi_effect value.

At step 620 of the method, the system compares the variant-target expression regulation information var_reg_results generated in steps 330-360 of the method to the gene-based CNV and epigenetic impact status from step 150 of the method. If the regulatory direction of the gene impacted by the variant is the same as the regulatory direction of that gene from the CNV and epigenetic impact status from step 150, then the variant-gene regulation entry is removed from the data structure var_reg_results.

At step 630 of the method, the adjusted variant-based expression regulation status and score are then computed by applying the same process 300 on the updated data structure var_reg_results.

At step 640 of the method, the system compares the gene-gene regulation information gene_reg_results generated in steps 440-450 of the method to the gene-based CNV and epigenetic impact status from step 150 of the method. If the regulatory direction of the gene impacted by the variant is the same as the regulatory direction of that gene from the CNV and epigenetic impact status from step 150, then the gene-gene regulation entry is removed from the data structure gene_reg_results.

At step 650 of the method, the adjusted gene-based expression regulation status and score are then computed by applying the same process 400 on the updated data structure gene_reg_results.

At step 660 of the method, the system generates a report comprising the finalized list of variants/genes and associated information on their expression regulatory effects after adjusting for relevant CNV and epigenetic factors. This can comprise storing the information in a data table or other data format, or via a printed or displayed report. According to an embodiment, the report comprises one or more of, for each variant:
adj_var_reg_status—a categorical variable that indicates the overall type of expression regulatory effect of a variant as “Cis-Promoter”, “Cis-Enhancer”, “Trans-eQTL”, “Cis and Trans” or “None”;
adj_var_reg_score—a score that measures the strength of gene expression regulatory evidence. It should be a function of var_reg_results, e.g. choosing the evidence_value with the largest magnitude (regardless of the sign/direction); and
adj_var_reg_results—a table that summarizes the specific variant-target regulatory effects with the following fields:
   type—the type of regulatory effect, which can be “Cis-Promoter,” “Cis-Enhancer,” “Trans-eQTL,” “Cis and Trans,” or “None”;
   target—the target of the regulatory action, which can be the symbol of the affected gene, optionally concatenated by “:” followed by the regulatory direction (up/down) if available;
   evidence_type—the type of supporting evidence for the regulatory effect, which can be “differential_gene_expression” or other applicable types; and/or
   evidence_value—a value that measures the strength of the supporting evidence. For evidence based on differential expression, it can be the log 2 fold change of case vs. control expression levels, among other values.
      And the following expression regulation information for each gene:
adj_gene_reg_status—a categorical variable that indicates the strongest type of expression regulatory effect of a gene on its direct gene targets defined in the pathway databases. Possible categories include “Agreed Direction”, “Unknown Direction”, “Non-DE”, “Opposite Direction” and “No Evidence”;
gene_reg_score_close—one or a vector of scores that quantify the overall evidence for expression regulatory effect of a gene on its immediate or close targets;
gene_reg_score_ext—one or a vector of scores that quantify the overall evidence for expression regulatory effect of a gene on its extended downstream targets, up to a user-defined distance d (in number of genes); and
gene_reg_results—a table that summarizes the specific gene-gene regulatory effects with the following fields:
   path_db:path—the pathway database and the gene pathway in which the gene-gene regulation is defined;
   gene_1—the upstream gene;
   gene_2—the direct downstream target gene;
   status—the type of evidence for the gene-gene regulation;
   de_1—differential expression value, e.g. in log 2 fold change, of gene_1;
   de_status_1—differential expression status, i.e. up/down, of gene_1;
   de_2—differential expression value, e.g. in log 2 fold change, of gene_2; and/or
   de_status_2—differential expression status, i.e. up/down, of gene_2.

Many other fields are possible. Although the values are associated with specific labels, it is appreciated that the labels may be any label. This information can comprise all or a portion of the report generated by the system at 170 of the method in FIG. 1.

At optional step 180 of the method, the system can filter and/or rank a plurality of variants and/or genes based at least in part on at least the adjusted variant-based expression regulation status and/or the adjusted gene-based expression regulation status information. For example, the system may use these or any other scores or statuses generated by the system to rank or score variants and/or genes. As one example, the system may create and report a list of genes and/or variants that are identified as comprising a particular effect, and rank them according to the likelihood of the potential strength of that impact. As another example, the system may create and report a list of only variants or genes that have an epigenetic effect, among many other potential lists or rankings.

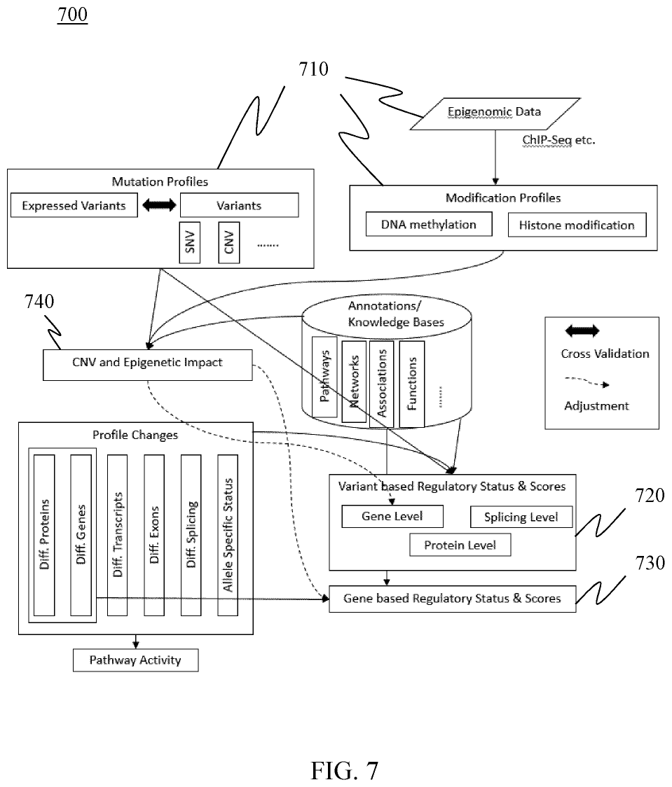

Referring to FIG. 7, in one embodiment, is a flowchart of a method 700 for characterizing variant expression status of variants in a genomic sample using a variant analysis system. The variant analysis system may be any of the systems described or otherwise envisioned herein, and may comprise any of the components described or otherwise envisioned.

At 710, the system receives information such as variant information, expression information, CNV information, epigenetic information, proteomic information, and/or any other information described or otherwise envisioned herein.

At 730, the system generates a splice status for a variant, the splice status comprising a type of splicing effect of the variant, and the system generates a variant-based expression regulation status and/or score. AT 740, the system generated a gene-based expression regulation status and/or score. At 740, the system determines the system generates a gene-based copy number variant (CNV) and/or epigenetic impact status and/or score. The system then utilizes the gene-based copy number variant (CNV) and/or epigenetic impact status and/or score to adjust the variant-based expression regulation status and/or score as well as the gene-based expression regulation status and/or score, as shown by the dotted lines.

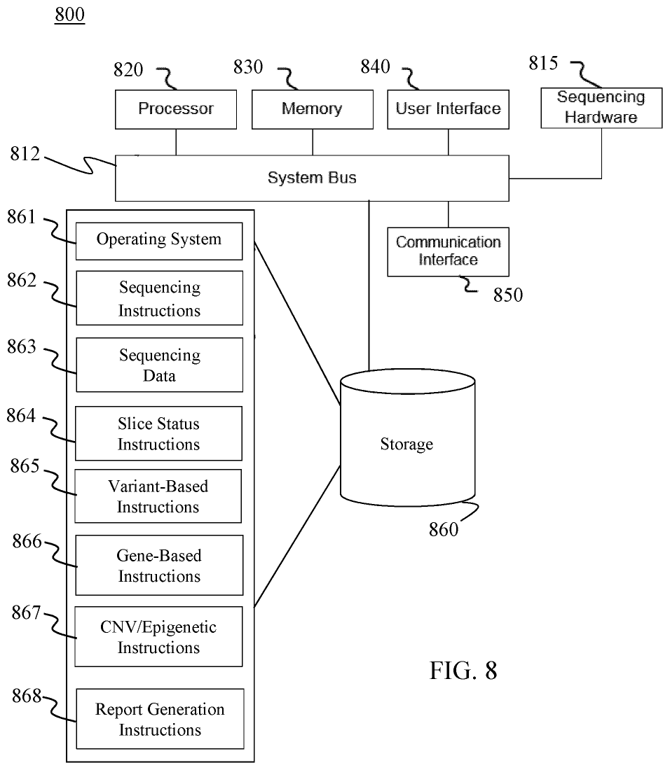

Referring to FIG. 8, in one embodiment, is a schematic representation of a variant analysis system 800 configured to characterize the functional impact of genomic variants identified from a genomic sample. System 800 may be any of the systems described or otherwise envisioned herein, and may comprise any of the components described or otherwise envisioned herein.

According to an embodiment, system 800 comprises one or more of a processor 820, memory 830, user interface 840, communications interface 850, and storage 860, interconnected via one or more system buses 812. In some embodiments, such as those where the system comprises or directly implements a DNA and/or RNA sequencer or sequencing platform, the hardware may include additional sequencing hardware 815. It will be understood that FIG. 8 constitutes, in some respects, an abstraction and that the actual organization of the components of the system 500 may be different and more complex than illustrated.

According to an embodiment, system 800 comprises a processor 820 capable of executing instructions stored in memory 830 or storage 860 or otherwise processing data to, for example, perform one or more steps of the method. Processor 820 may be formed of one or multiple modules. Processor 820 may take any suitable form, including but not limited to a microprocessor, microcontroller, multiple microcontrollers, circuitry, field programmable gate array (FPGA), application-specific integrated circuit (ASIC), a single processor, or plural processors.

Memory 830 can take any suitable form, including a non-volatile memory and/or RAM. The memory 830 may include various memories such as, for example L1, L2, or L3 cache or system memory. As such, the memory 830 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 800. It will be apparent that, in embodiments where the processor implements one or more of the functions described herein in hardware, the software described as corresponding to such functionality in other embodiments may be omitted.

User interface 840 may include one or more devices for enabling communication with a user. The user interface can be any device or system that allows information to be conveyed and/or received, and may include a display, a mouse, and/or a keyboard for receiving user commands. In some embodiments, user interface 840 may include a command line interface or graphical user interface that may be presented to a remote terminal via communication interface 850. The user interface may be located with one or more other components of the system, or may located remote from the system and in communication via a wired and/or wireless communications network.

Communication interface 850 may include one or more devices for enabling communication with other hardware devices. For example, communication interface 850 may include a network interface card (NIC) configured to communicate according to the Ethernet protocol. Additionally, communication interface 850 may implement a TCP/IP stack for communication according to the TCP/IP protocols. Various alternative or additional hardware or configurations for communication interface 850 will be apparent.

Storage 860 may include one or more machine-readable storage media such as read-only memory (ROM), random-access memory (RAM), magnetic disk storage media, optical storage media, flash-memory devices, or similar storage media. In various embodiments, storage 860 may store instructions for execution by processor 820 or data upon which processor 820 may operate. For example, storage 860 may store an operating system 861 for controlling various operations of system 800. Where system 800 implements a sequencer and includes sequencing hardware 815, storage 860 may include sequencing instructions 862 for operating the sequencing hardware 815, and sequencing data 863 obtained by the sequencing hardware 815, although sequencing data 863 may be obtained from a source other than an associated sequencing platform.

It will be apparent that various information described as stored in storage 860 may be additionally or alternatively stored in memory 830. In this respect, memory 830 may also be considered to constitute a storage device and storage 860 may be considered a memory. Various other arrangements will be apparent. Further, memory 830 and storage 860 may both be considered to be non-transitory machine-readable media. As used herein, the term non-transitory will be understood to exclude transitory signals but to include all forms of storage, including both volatile and non-volatile memories.

While variant analysis system 800 is shown as including one of each described component, the various components may be duplicated in various embodiments. For example, processor 820 may include multiple microprocessors that are configured to independently execute the methods described herein or are configured to perform steps or subroutines of the methods described herein such that the multiple processors cooperate to achieve the functionality described herein. Further, where one or more components of system 800 is implemented in a cloud computing system, the various hardware components may belong to separate physical systems. For example, processor 820 may include a first processor in a first server and a second processor in a second server. Many other variations and configurations are possible.

According to an embodiment, storage 860 of variant analysis system 800 may store one or more algorithms and/or instructions to carry out one or more functions or steps of the methods described or otherwise envisioned herein. For example, processor 820 may comprise splice status instructions or software 864, variant-based expression regulation status instructions or software 865, gene-based expression regulation status instructions or software 866, gene-based CNV epigenetic impact status instructions or software 867, and/or report generation instructions or software 868, among many other algorithms and/or instructions to carry out one or more functions or steps of the methods described or otherwise envisioned herein.

According to an embodiment, splice status instructions or software 864 direct the system to generate a splice status for one or more variants, the splice status comprising a type of splicing effect of the variant. This is effectively a variant-based splicing regulatory status and score. The splice status may comprise a cis and trans splicing effect indicating that the variant affects splicing of a local and a distant gene.

According to an embodiment, variant-based expression regulation status instructions or software 865 direct the system to generate a variant-based expression regulation status. This is effectively an analysis of the variant on the regulation of expression of one or more cis and/or trans genes. For example, the variant-based expression status may comprise a predefined or user-defined variable that indicates that the variant has no effect on the regulation of expression, upregulation of a cis and/or trans gene, and/or downregulation of a cis or trans gene, among other indications.

According to an embodiment, gene-based expression regulation status instructions or software 866 direct the system to generate a gene-based expression regulation status. This is effectively an analysis of gene-gene interactions to determine whether a variant has a functional impact on a target gene in a pathway. For example, the gene-based expression regulation status may comprise a variable that indicates the strongest type of expression regulatory effect of a gene on its direct gene targets defined in the pathway databases. According to an embodiment, the goal is to evaluate the functional evidence for each gene-gene interaction as identified by differential expression and as defined in external pathway databases.

According to an embodiment, gene-based CNV epigenetic impact status instructions or software 868 direct the system to generate a gene-based copy number variant (CNV) and/or epigenetic impact status and/or score. This is an analysis of the CNV and epigenetic influence on each gene. For example, gene-based copy number variant (CNV) and epigenetic impact status may comprise a categorical value that indicates the combined CNV and epigenetic effect on the gene expression. According to an embodiment, the goal is to the CNV and epigenetic influence on each gene.

According to an embodiment, report generation instructions or software 869 direct the system to generate a user report comprising information about the analysis performed by the system. For example, a report may comprise the finalized list of variants and associated information generated by the method and system. The report may be generated for any format or output method, such as a file format, a visual display, or any other format. A report may comprise a text-based file or other format comprising the reported information.

The report generation instructions or software 868 may direct the system to store the generated report or information in temporary and/or long-term memory or other storage. This may be local storage within system 800 or associated with system 800, or may be remote storage which received the report or information from or via system 800. Additionally and/or alternatively, the report or information may be communicated or otherwise transmitted to another system, recipient, process, device, and/or other local or remote location.

The report generation instructions or software 868 may direct the system to provide the generated report to a user or other system. For example, the system may visually display information about one or more of the variants on the user interface, which may be a screen or other display. A clinician or researcher may only be interested in one or several variants, and thus the variant analysis system may be instructed or otherwise designed or programmed to only display information obtained for the one or several variants.

One major challenge in genomic research and precision medicine is to identify the mutations and/or genes that actually cause disease symptoms, out of the hundreds and thousands of candidate variants, which is necessary for scientific discovery or identification of potential treatment targets. While standard variant-filtering approaches based on call quality, population allele frequency, gene-model annotation, known disease association, and predicted pathogenicity can narrow down the pool of candidate variants, multi-omic data analysis of gene expression, CNV, epigenetic, and other data is critical for explaining further the molecular mechanism(s) of disease, which sheds light on disease etiology and treatment options.

One use case of the multi-omic data analysis framework described or otherwise envisioned herein is to facilitate the discovery of causal variants of a disease by performing analysis on the DNA and RNA whole exome sequencing (WES) data of hundreds of samples in a genomic study. By comparing the exon/gene/transcript expression between the carrier and non-carriers of each candidate variant, and using external databases (e.g. expression/splicing quantitative trait loci, promoter/enhancer map, etc.), our framework can evaluate whether a variant has any impact on allele-specific expression, alternative splicing, regulation of target genes, etc. The generated variant-based statuses and scores, as described herein, can then be used to filter and rank variants by their potential functional impacts.

In addition to variant-based functional impact evaluation, scientists may also gain insights on the functional impact of individual genes. This can be done using the framework described or otherwise envisioned herein to analyze the differential gene expressions between the case and control samples. With reference to pathway definitions in external databases such as KEGG, Reactome and Pathway Commons, the framework can evaluate whether a gene has any impact on its immediate/nearby downstream target genes or overall pathway activities. If CNV, methylation, or other epigenetic data are available, the framework can evaluate the combined CNV and epigenetic impact on each gene. This, in combination with the gene expression results, can further indicate if the differential expression of a gene or any regulatory effect is indeed driven by CNV or epigenetic factors. By carefully and systematically considering the multi-layer evidence obtained from the different -omic data, scientists can pinpoint the causal mutations with explanations for their potential influence on gene targets and pathways.

In a similar fashion, clinicians can use the framework described or otherwise envisioned herein to analyze the DNA and RNA WES data to identify the causal disease mutations or genes in a patient. When evaluating variant-based functional impact, if the data of one patient is insufficient, the gene expression data of carriers and non-carriers from other studies can be employed. Using the framework described or otherwise envisioned herein, clinicians can pinpoint the causal mutations and genes with explanations for the molecular mechanism. For example, if a disease is found to be caused by a gene mutation that leads to the up-regulation of the activity of a pathway, then a drug known to suppress the activity of the pathway can be administered to the patient in an attempt to cure the disease or alleviate the symptoms.

Thus, according to an embodiment, the methods and systems described or otherwise envisioned herein comprise many different practical applications. For example, the output of the system or method may be a report comprising one or more of the characterized plurality of statuses and/or scores including a splice status, a variant-based expression regulation status and/or score, a gene-based expression regulation status and/or score, and a gene-based CNV and epigenetic impact status and/or score, among other reports, statuses, and information. This report has many uses, including being used by a physician or other healthcare professional, or a researcher, to determine genes and/or variants involved in the phenotype of a particular individual such as a cancer patient or sufferer or a rare genetic disease, among many other possible individuals. The system may generate a report that not only includes a list of genes and/or variants likely to be involved in the phenotype of a particular individual, but the report may also comprise a ranking of the most likely genes and/or variants, and/or a ranking of the largest impact of likely genes and/or variants, and/or a ranking of genes and/or variants with the most supporting evidence for impact.

Accordingly, methods and systems described or otherwise envisioned herein further comprise the step of receiving, a scientist, healthcare professional or other individual, a report generated by the system and comprising any of the information described or otherwise envisioned herein. The receiving individual reviews the report and identifies one or more genes and/or variants identified in the report as being likely to be involved in the test-taker's phenotype, and therefore likely targets for treatment and/or intervention. According to one embodiment, once an identification is made the receiving individual or a person acting on behalf of the receiving individual implements a treatment or intervention to treat the phenotype. This may include a specific medical treatment based on a known association between the identified variant and/or genes and specific medicines or interventions, for example. According to another embodiment, once an identification is made the receiving individual or a person acting on behalf of the receiving individual can utilize the information for research purposes to identify potential treatment and/or interventions. Thus there can be a direct relationship between the variant and genes, the output of the analytical method and system that examines the variant and genes, and the treatment or study of the individual.

The methods and systems described herein comprise several limitations each comprising and analyzing millions of pieces of information. For example, the variant information and associated expression (and potentially other) information received or generated by the system likely comprises many 1000s of potential variants, genes, and other points of data for analysis. Similarly, each step of the process comprises analysis of those 1000s of potential variants, genes, and other points of data, thereby constituting millions of calculations. This is something the human mind is not equipped to perform, even with pen and pencil.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for characterizing a functional impact of a plurality of variants identified from a genomic sample, using a variant analysis system, comprising:
obtaining genomic sample information, the genomic sample information comprising at least a plurality of variants identified in the genomic sample, gene expression information obtained from the genomic sample, copy number variation for one or more genes in the genomic sample, and epigenetic effects on one or more genes in the genomic sample;
determining a splice status for the variant, the splice status comprising an indication of whether a variant has an effect on splicing of a gene;
determining a variant-based expression regulation status, the variant-based expression regulation status comprising an indication of whether the variant has an effect on expression of a gene;
determining a gene-based expression regulation status, the gene-based expression regulation status comprising an indication of whether the variant has a functional impact on a target gene in a pathway;
determining a gene-based copy number variant (CNV) and epigenetic impact status, the gene-based CNV and epigenetic impact status comprising an indication of whether the CNV and/or epigenetic impact has an impact on expression of a gene;
adjusting, based on the gene-based CNV and epigenetic impact status, the variant-based expression regulation status and/or the gene-based expression regulation status; and
reporting at least the adjusted variant-based expression regulation status and/or the adjusted gene-based expression regulation status for each of a plurality of variants and/or genes in the genomic sample.

2. The method of claim 1, further comprising the step of filtering at least some of the plurality of variants or genes based at least on the adjusted variant-based expression regulation status and/or the adjusted gene-based expression regulation status associated with each respective variant and/or gene.

3. The method of claim 1, further comprising the step of ranking at least some of the plurality of variants or genes.

4. The method of claim 1, wherein the splice status further comprises an indication of a strength of splicing evidence for the effect on splicing of the gene.

5. The method of claim 1, wherein the variant-based expression regulation status further comprises an indication of whether the affected gene is local or remote.

6. The method of claim 1, wherein the gene-based expression regulation status further comprises an indication of whether the target gene is upregulated or downregulated.

7. The method of claim 1, wherein the gene-based copy number variant (CNV) and epigenetic impact status further comprises an indication of whether the copy number variant (CNV) and/or epigenetic impact results in upregulation or downregulation of a gene.

8. The method of claim 7, wherein reporting comprises a table or other data structure comprising a list of variants and/or genes and the functional impact information associated with each variant and/or gene.

9. The method of claim 8, wherein the functional impact information comprises, for one or more of the plurality of remaining variants, an indication of an effect of the variant on the expression of one or more genes.

10. A system for characterizing a functional impact of a plurality of variants identified from a genomic sample, comprising:
genomic sample information, the genomic sample information comprising at least a plurality of variants identified in the genomic sample, gene expression information obtained from the genomic sample, copy number variation for one or more genes in the genomic sample, and epigenetic effects on one or more genes in the genomic sample; and
a processor configured to: (i) determine a splice status for the variant, the splice status comprising an indication of whether a variant has an effect on splicing of a gene; (ii) determine a variant-based expression regulation status, the variant-based expression regulation status comprising an indication of whether the variant has an effect on expression of a gene; (iii) determine a gene-based expression regulation status, the gene-based expression regulation status comprising an indication of whether the variant has a functional impact on a target gene in a pathway; (iv) determine a gene-based copy number variant (CNV) and epigenetic impact status, the gene-based CNV and epigenetic impact status comprising an indication of whether the CNV and/or epigenetic impact has an impact on expression of a gene; and (v) adjust, based on the gene-based CNV and epigenetic impact status, the variant-based expression regulation status and/or the gene-based expression regulation status; and
a user interface configured to report at least the adjusted variant-based expression regulation status and/or the adjusted gene-based expression regulation status for each of a plurality of variants and/or genes in the genomic sample.

11. The system of claim 10, wherein the processor is further configured to filter at least some of the plurality of variants or genes based at least on the adjusted variant-based expression regulation status and/or the adjusted gene-based expression regulation status associated with each respective variant and/or gene.

12. The system of claim 10, wherein the adjusted variant-based expression regulation status and/or the gene-based expression regulation status comprises a table or other data structure comprising a list of variants and/or genes and functional impact information associated with each variant and/or gene.

13. The system of claim 12, wherein the wherein the functional impact information comprises, for one or more of the plurality of remaining variants, an indication of an effect of the variant on the expression of one or more genes.

14. The system of claim 10, wherein the variant-based expression regulation status further comprises an indication of whether the affected gene is local or remote.

15. The system of claim 10, wherein the gene-based expression regulation status further comprises an indication of whether the target gene is upregulated or downregulated.
